(19) United States
(12) Patent Application Publication
(10) Pub. No.: US 2015/0164990 A1
(43) Pub. Date: Jun. 18, 2015

(54) Compositions and Methods for Preventing and/or Treating Sensorineural Hearing Loss

(71) Applicants: Marilyn Sandra Geaney, Papatoetoe (NZ); Robert Keith Shepherd, Ascot Vale (AU); Andrew Wise, East Melbourne (AU); James Fallon, East Melbourne (AU)

(72) Inventors: Marilyn Sandra Geaney, Papatoetoe (NZ); Robert Keith Shepherd, Ascot Vale (AU); Andrew Wise, East Melbourne (AU); James Fallon, East Melbourne (AU)

(73) Assignees: The Bionic Ear Institute, East Melbourne (AU); Living Cell Technologies Limited, Sydney (AU)

(21) Appl. No.: 14/109,763

(22) Filed: Dec. 17, 2013

(51) Int. Cl. A61K 38/18; A61K 9/00; A61K 35/30

(52) U.S. Cl. CPC A61K 38/185; A61K 35/30; A61K 9/0046

(57) ABSTRACT

The present invention is directed to the prevention or treatment of sensorineural hearing loss by administering a therapeutically effective amount of an implantable composition comprising encapsulated living choroid plexus cells.

### CROSS-REFERENCE TO RELATED APPLICATIONS

The present application is a Continuation of U.S. patent application Ser. No. 12/498,887, filed Jul. 7, 2009 (co-pending; Atty. Dkt. No. 36697.28); which is a Continuation-in-Part of Ser. No. 11/682,810, filed Mar. 6, 2007 (abandoned; Atty. Dkt. No. 36697.21); which is a Continuation of U.S. Provisional Patent Application No. 60/866,811, filed Nov. 21, 2006 (abandoned). The entire content of each of these applications is specifically incorporated herein in its entirety by express reference thereto.

### FIELD OF THE INVENTION

The present invention relates generally to the fields of biology and medicine. Particular aspects of the invention are directed to compositions and methods for the therapy and/or prophylaxis of various types of hearing loss in an animal, particularly although by no means exclusively to the prevention and/or treatment of hearing loss attributable to degeneration, defect, or dysfunction of all or part of the auditory nerve.

### BACKGROUND

Hearing loss is the most prevalent disability in the world. The World Health Organization estimates 250 million people world-wide currently suffer from a disabling hearing impairment and predict this number will continue to increase. This is due partly to the incidence of new cases—approximately 4,000 new cases of sudden deafness occur each year in the United States, and partly to an aging population. For example, the proportion of people with a hearing loss rises from approximately 30% of people over age 65, to 40-50% of people 75 and older, to nearly 90% of people over age 80.

An inability to hear properly, or at all, can have detrimental effects on children and adults alike. In children, hearing loss can impair language development and communication skills, thus leading to difficulties in social and learning situations. In addition to affecting their sense of well-being, deafness in adults can have serious effects on a person's ability to be employed and to interact socially. While hearing aids, which amplify sound, are helpful for those with some forms of hearing loss, they are not useful in treating the permanent, severe-profound deafness experienced with sensorineural hearing loss (SNHL).

SNHL accounts for about 90% of all hearing loss. SNHL is due to damage to either the cochlea or the auditory nerve. Common causes include old age, where the hearing pattern is often called presbyacusis, Ménière's disease, ototoxic medications (such as high-dose aspirin or certain strong diuretics), immune disorders, and noise exposure. Trauma, including inner ear concussion, can cause both temporary and permanent hearing loss.

Currently, SNHL is treated with hearing aids, which amplify sounds at pre-set frequencies to overcome a SNHL in that range, or with cochlear implants, which stimulate the cochlear nerve directly.

A cochlear implant is a surgically implanted electronic device that can help provide a sense of sound to a person who is profoundly deaf or severely hard of hearing. Unlike other kinds of hearing aids, the cochlear implant doesn't amplify sound, but works by directly stimulating any functioning auditory nerves inside the cochlea. The cochlear implant usually comprises external components, including a microphone, speech processor and transmitter.

An implant does not restore or create normal hearing. Instead, under the appropriate conditions, an implant may give a deaf person a useful auditory understanding of the environment and help them to understand speech. Post-implantation therapy may also be required.

For those with a profound SNHL, the actual benefits of cochlear implantation using currently available implants vary widely. This is at least in part because the implant works by stimulating the spiral ganglion neurons (SGNs) of the auditory nerve, and thus requires the presence of some functioning auditory nerve cells.

With many SNHLs, the degeneration of the affected neurons is ongoing, so that any treatment has to continue for the lifetime of the patient.

It has been reported that delivery of neurotrophic factors, such as brain derived neurotrophic factor (BDNF), and neurotrophic factor 3 (NT-3), to the cochlea improves the survival of SGNs (reviewed in Marzella and Gillespie, 2002). This effect can reportedly be potentiated with electrical stimulation, such as that provided by the cochlear implant (Shepherd, R K, et al., 2005). Neurotrophins have also been reported to cross the round window membrane and protect SGNs from degeneration following ototoxin induced deafness (Noushi F, et al., 2005). Unfortunately, the observed neurotrophin-induced survival effects are reportedly lost if the neurotrophic treatment is withdrawn (Gillespie, L N, et al., 2003).

Cell-based therapies have been investigated as a means of supporting auditory neuron survival in deafness. A review of such therapies is presented elsewhere (Gillespie L K and Shepherd R K, 2005). For example, it has been reported that Schwann cells can prevent deafness-induced auditory neuron degeneration in vivo (Andrew, J K, 2005).

A disadvantage of many cell-based therapies is the introduction of foreign matter into the patient and thus the requirement for immunosuppression to prevent rejection of the foreign matter. A further disadvantage of current cell-based therapies is the less than optimal level of production or secretion of desired neurotrophins. Also, delivery of individual cells into the cochlea is known to result in widespread dispersal and loss of cells from the cochlea reducing therapeutic efficacy (Coleman, B, et al., 2006).

There remains a need for a method to enable continuous treatment for long-term or permanent rescue of SGNs from degeneration, and so to treat or prevent hearing loss.

It is therefore desirable to provide a method for treating hearing loss in patients with or at risk of developing SNHL. It would also be desirable if such a method could also be used to prevent hearing loss in patients with or at risk of developing SNHL.

It is an object of the invention to go some way towards achieving these desiderata and/or to provide the public with a useful choice.

### SUMMARY

In a first embodiment, the present invention provides a method for reversing, preventing or delaying the degeneration of auditory cells in a patient at risk thereof. The method in a general sense involves at least the step of implanting in such a patient a composition that comprises at least a first population of encapsulated living choroid plexus (CP) cells.

The present invention further provides a method for treating sensorineural hearing loss in a patient in need thereof. This method, in an overall and general sense involves at least the step of implanting in such a patient a composition that comprises encapsulated living choroid plexus cells.

In another aspect, the present invention also provides a use of at least a first population of encapsulated living choroid plexus cells in the manufacture of an implantable composition to reverse, prevent or delay the degeneration of auditory cells in a patient in need thereof.

The present invention further provides a use of encapsulated living choroid plexus cells in the manufacture of an implantable composition to treat sensorineural hearing loss in a patient in need thereof.

Additionally, the present invention further provides an implantable device that comprises at least a first population of encapsulated living choroid plexus cells for use in the treatment of sensorineural hearing loss in a patient in need thereof.

Another aspect of the present invention provides an implantable device that comprises at least a first population of encapsulated living choroid plexus cells. In one embodiment, the implantable device is selected for implantation into a patient to reverse, prevent and/or delay the degeneration of auditory cells in such a patient.

The encapsulated living choroid plexus cells will preferably be implanted in an amount sufficient to secrete a therapeutically-effective amount of neurotrophin factors. The encapsulated choroid plexus cell implants may be used in the present invention in combination with one or more additional therapies, including, for example, one or more traditional therapies for sensorineural hearing loss. Likewise the implant may also be employed in combination with a cochlear implant and/or in combination with one or more therapeutics, or neurotrophic factors (including, for example, but not limited to TGFβ, IGFM, VEGF, NT, NGF, FGF, EGF etc.

The choroid plexus cells may also be combined with one or more other neurotrophin-secretory cells such as Schwann cells, retinal pigmented epithelium, dorsal root ganglia, or other cells as described herein. Alternatively or additionally, the CP cells may be implanted with one or more feeder cells or support cells to increase the viability of the implantable composition. Examples of feeder cells or support cells include, for example, but are not limited to Sertoli cells, fibroblasts, splenocytes, thymocytes, etc, again as described herein.

It is also contemplated that encapsulated choroid plexus cells can be used to reverse, prevent or delay the onset of degeneration of other cells associated with the middle or inner ear, the cochlea or the auditory nerve, such as hair cells, cochlear epithelial cells, cells of the scala tympani, supporting cells of the organ of Corti, endogenous Schwann cells, other transplanted cells, and the like.

The neurotrophin-secretory cells preferably have a neurotrophic factor secretory profile, more preferably a neurotrophic factor secretory profile that is functionally equivalent to that of choroid plexus cells. Such cells may be naturally-occurring, or may be genetically engineered to express one or more neutrophins.

### BRIEF DESCRIPTION OF THE DRAWINGS

For the purpose of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

Aspects of the invention will now be described in more detail by reference to the following figures in which,

FIG. 1 and FIG. 2 show encapsulated choroid plexus cells, and encapsulated Schwann cells, prepared as described herein in Examples 2 and 3, respectively, infra.

FIG. 3A and FIG. 3B shows the implantation of microcapsules prepared as described herein into the cochlea of an animal model of SNHL as described in Example 4.

FIG. 4A, FIG. 4B, and FIG. 4C is are photomicrographs illustrating the histological analysis of the site of implantation of microcapsules implanted into the cochlea of an animal model of SNHL as described herein in Example 5. FIG. 4B and FIG. 4C are magnified images of the identified areas depicted in FIG. 4A, showing the disposition of neurons (e.g., rendering them suitable for neuronal counting), and the location of the microcapsules within the cochlea, respectively.

FIG. 5A, FIG. 5B, and FIG. 5C are photomicrographs illustrating the surgical delivery of microcapsules to the cochlear of an animal model of SNHL in which a cochlear electrode array device had already been implanted, as described in Example 9 herein. FIG. 5B is a magnified image of the dotted region shown in FIG. 5A, while FIG. 5C shows the implanted cochlear electrode array device in situ with the implanted capsules.

FIG. 6A, FIG. 6B, and FIG. 6C are graphs presenting SGN density data at 4 regions of the cochlear (lower basal (LB), upper basal (UB), lower middle (LM), and upper middle (UM)) for each of the three experimental groups as described in Example 10 herein. FIG. 6A shows SGN density data for the experimental group receiving chronic ICES alone, FIG. 6B shows SGN density data for the experimental group receiving implanted encapsulated choroid plexus cells alone, and FIG. 6C shows SGN density data for the experimental group receiving both implanted encapsulated choroid plexus cells and chronic ICES, as described in Example 10 herein.

FIG. 7 is a graph presenting electrically evoked auditory brainstem response (EABR) thresholds for the three treatment groups described herein in Example 10. EABR thresholds were averaged across the seven electrodes of the intracochlear electrode array for each animal. This average EABR threshold was then represented as a percentage of the value measured in the first recording for each experimental condition and was plotted against treatment period.

### DESCRIPTION OF ILLUSTRATIVE EMBODIMENTS

The present invention recognizes the capacity of cell-based delivery of neurotrophins to provide the long-term rehabilitation of spiral ganglion neurons (SGNs) of the auditory nerve following degeneration caused by or resulting in sensorineural hearing loss (SNHL).

The present invention further recognizes that living choroid plexus cells can be useful in reversing, preventing or delaying auditory cell degeneration. Choroid plexus cells have not, previously, been linked to auditory function.

The present invention is directed to a method for reversing, preventing or delaying auditory cell degeneration by administering a therapeutically effective amount of implantable composition comprising encapsulated living choroid plexus cells to a patient in need thereof.

The present invention is further directed to a method of treating sensorineural hearing loss by administering a therapeutically effective amount of an implantable composition comprising encapsulated living choroid plexus cells to a patient in need thereof.

The composition may additionally comprise other cell types, such as, for example, cells able to provide one or more trophic factors or functions to the choroid plexus cells, such as support cells or feeder cells, or other neurotrophin-secreting cells.

Neurotrophins are protective hormones and proteins that have a range of trophic effects on cellular growth, repair and function, and generally encourage the survival of nerve tissues. Examples of neurotrophins include transforming growth factor β1, β2, β3, and β5, (TGFβ1, TGFβ2, TGFβ3, TGFβ5, respectively), growth/differentiation factor-15 (GDF-15), glial cell derived neurotrophic factor (GDNF), insulin-like growth factor 1 (IGF-1), insulin-like growth factor 2 (IGF-2), insulin-like growth factor receptor (IGF-R), nerve growth factor (NGF), neurotrophin 3 (NT-3), neurotrophin 4 (NT-4), neurotrophin 5 (NT-5), brain derived growth factor (BDNF), vascular endothelial growth factor (VEGF), and fibroblast growth factor 2 (FGF2). The role of various neurotrophins in the development, survival and repair of auditory neurons is reviewed elsewhere (Marzella and Gillespie, 2002). Other neurotrophins implicated in the development and maintenance of auditory neurons include epidermal growth factor (EGF), epidermal growth factor receptor (EGFR), fibroblast growth factor receptor 2 (FGFR-2, [IIIb isoform]), fibroblast growth factor receptor 3 (FGFR-3), ciliary-derived neurotrophic factor (CNTF), leukemia inhibitory factor (LIF), TrkB, TrkC, and p75.

Choroid plexus cells are cells capable of expressing and secreting a particular profile of neurotrophins that are useful in the treatment and prevention of hearing loss. Additional neurotrophin-secretory cells may be used in combination with CP cells to treat and or prevent hearing loss including cells having a neuronal factor secretory profile that is functionally equivalent to that of choroid plexus cells. Examples of such additional neurotrophin secretory cells include Schwann cells, and cells genetically engineered to express one or more neutrophins.

Choroid plexus cells are isolated from the choroid plexus, lobulated structures comprising a single continuous layer of cells derived from the ependymal layer of the cerebral ventricles. One function of the choroid plexus is the secretion of cerebrospinal fluid (CSF). Cerebrospinal fluid fills the four ventricles of the brain and circulates around the spinal cord and over the convexity of the brain. The CSF is continuous with the brain interstitial (extracellular) fluid, and solutes, including macromolecules, are exchanged freely between CSF and interstitial fluid. In addition to the production of CSF, the choroid plexus has been associated with the formation of the CSF-blood barrier (Aleshire, S L, et al., 1985). However, its broader function is the establishment and maintenance of baseline levels of the extracellular milieu throughout the brain and spinal cord, in part by secreting a wide range of growth factors into the CSF. Studies have reported the presence of numerous potent trophic factors within choroid plexus including TGF-β, GDF-15, GDNF, IGF2, NGF, NT-3, NT-4, BDNF, VEGF, and FGF2 (for review see e.g., Johanson, C E, et al., 2000). However, to date the CP secreted factors have not been thought to be useful in preventing or treating hearing loss. Preferred neurotrophin-secretory cells include cells having a neurotrophic factor secretory profile functionally equivalent to that of choroid plexus cells, and include Schwann cells, retinal-pigmented epithelium, dorsal root ganglia, and cells genetically engineered to express one or more neutrophins.

CP cells may be used in combination with additional neurotrophin-secretory cells, preferably Schwann cells. Schwann cells are a variety of neuroglia, and comprise myelinating Schwann cells and non-myelinating Schwann cells. Myelinating Schwann provide myelin insulation to axons in the peripheral nervous system, decreasing membrane capacitance in the axon and allowing signal conduction to occur and for an increase in impulse speed without an increase in axonal diameter. Non-myelinating Schwann cells are involved in maintenance of axons and are crucial for neuronal survival. Schwann cells secrete neurotrophins, such as brain-derived neurotrophin (BDNF), a low molecular mass (14 kDa, or 27 kDa as the dimer) neurotrophin that stimulates and nurtures neuronal cells.

Yet further preferred neurotrophin-secretory cells are cells, such as Schwann cells, genetically engineered to express and secrete one or more neurotrophins. Many such cells have been described, including, for example, Schwann cells genetically engineered to overexpress and secrete BDNF (see, e.g., Example 2 herein, and Sayers, S T, et al., 1998). The neurotrophins secreted by these genetically engineered cells may be naturally occurring neurotrophins or recombinant neurotrophins that are functionally equivalent to naturally occurring neurotrophins. As used herein, a functionally equivalent neurotrophin will elicit at least one biological effect elicited by the naturally occurring neurotrophin to which it is functionally equivalent.

The choroid plexus cells (and indeed the neurotrophin-secretory cells or support or feeder cells) may be from the same species as the host recipient patient, i.e., allograft, or may be from a different species, i.e., xenograft. In some embodiments, one or more of the cell types to be implanted (e.g., the Schwann cells), may be autologous. A preferred source of choroid plexus cells for clinical use is from bovine or porcine donors or cell lines. In certain embodiments, a preferable source of the choroid plexus cells is from porcine donors and in particular, from the Auckland Island herd of pigs. These pigs are substantially microorganism-free, and in particular have a very low porcine endogenous retrovirus (PERV) copy number, making them highly suitable as donors for xenotransplantation (Garkavenko, O, et al., 2004).

For example, the choroid plexus cell may be obtained from embryonic (fetal), newborn (neonatal) and adult pigs. Preferably, the choroid plexus cells are isolated from pigs aged from −20 to +20 days old.

For example, neonatal choroid plexus cells will be generally be preferred for xenotransplantation as their isolation is typically less problematic than their fetal counterparts, whilst their survival following isolation, for example, in tissue culture or following xenotransplantation, is commonly better than adult choroid plexus cells. For pigs, the neonatal period is generally held to be the first 7 to 21 days following birth.

Typically, embryonic porcine cells are isolated during selected stages of gestational development. For example, cells can be isolated from an embryonic pig at a stage of embryonic development when the cells can be recognized, or when the degree of growth and/or differentiation of the cells is suitable for the desired application. For example, the cells are isolated between about day twenty to about day twenty-five of gestation and birth of the pig.

The isolated choroid plexus cells for use in the invention can be maintained as a functionally viable cell culture. Examples of the methods by which choroid plexus cells can be cultured include, but are not limited to, those methods presented in PCT. Intl. Pat. Appl. Publ. Nos. WO 01/52871; WO 02/32437; WO 2004/113516; WO 03/027270; and WO 00/66188, and/or New Zealand Pat. Appl. Nos. NZ 532057, NZ 532059, and NZ 535131, each of which is specifically incorporated herein by reference in their entirety). Media which can be used to support the growth of porcine cells include, but are not limited to mammalian cell culture media (e.g., Dulbecco's minimal essential medium [DMEM]), and minimal essential medium [MEM]). The medium can be serum-free but is preferably supplemented with animal serum such as fetal calf serum, or more preferably, porcine serum (i.e., autologous serum). As will be appreciated by those skilled in the art, culture methods and conditions can be varied depending on the cell type to optimize cell growth and viability, neurotrophin production and secretion and maintenance of a neurotrophin-secreting phenotype.

The isolated choroid plexus cells may be co-cultured with neurotrophin-secretory cells, and/or with feeder cells or support cells, such as fibroblasts, Sertoli cells, splenocytes, thymocytes, etc. Such support or feeder cells secrete growth factors which enhance the viability of the neurotrophin-secretory cells.

The feeder cells or support cells may be isolated from the same donor as the choroid plexus cells.

The implantable compositions used in the present invention may comprise a combination of choroid plexus cells and one or more types of neurotrophin-secretory cells, feeder cells or support cells. It is envisaged that such a composition will remain viable in vivo for sustained periods of time.

When isolated from a donor, for example a donor pig, or taken from a cell line, the choroid plexus cells used in the invention retain their phenotype and/or are capable of performing their function. Preferably, isolated choroid plexus cells are capable of maintaining differentiated functions in vitro and in vivo, and of adhering to substrates, such as culture dishes. Similarly, the isolated neurotrophin-secretory cells, feeder cells or support cells are preferably capable of maintaining differentiated functions in vitro and in vivo, and of adhering to substrates, such as culture dishes.

The implantable composition comprises living choroid plexus cells (together with any pharmaceutically acceptable carriers or excipients) encapsulated in a biocompatible hydrogel such as alginate. Methods for the isolation and encapsulation of choroid plexus cells are described herein and elsewhere. For example, isolation and encapsulation of choroid plexus cells in alginate is described, inter alia, in PCT. Intl. Pat. Appl. Publ. No. WO 00/66188 (specifically incorporated herein by reference in its entirety). Preferably, the living choroid plexus cells are encapsulated in alginate. Such encapsulation acts to protect the choroid plexus cells from destruction by the recipient host's immune system. Exemplary methods to encapsulate choroid plexus cells to produce an implantable composition in accordance with the present invention are described herein in the Examples.

The implantable composition may also comprise other cells capable of secreting neurotrophins, and such neurotrophin-secretory cells may be encapsulated separately or together with the choroid plexus cells.

The implantable composition may further comprise “naked” living feeder cells or support cells, or the feeder cells or support cells may be encapsulated separately or together with the choroid plexus cells.

The implantable composition may additionally comprise, or be implanted with, neurotrophic factors, including one or more neurotrophins as described herein. These neurotrophic factors can be used to support the encapsulated cells while they become established at the implantation site.

Preferably the implantable composition for use in the methods of the present invention comprises alginate capsules of approximately 100 to 700 microns in diameter and containing approximately 1 to 3,000 living choroid plexus cells per capsule. Capsules of varying size can be produced by varying the encapsulation conditions, for example as described herein. The cochlea is a comparatively small target site for implantation compared to other sites commonly used for implantation of therapeutic implants. Moreover, the present invention recognizes that the internal structure of the inner ear, cochlea and supporting structures and their function constrain the design of the implantable composition to be implanted, and that in some applications capsules of varying size are beneficial to achieving an optimal therapeutic affect. Accordingly, capsules of about 100, about 150, about 200, about 250, about 300, about 350, about 400, about 450, about 500, about 550 microns, or any range therein, in diameter are contemplated for use in the present invention. When feeder cells or support cells are present, the capsules will contain approximately 500-3,000 living feeder cells or support cells or will contain 500-3,000 feeder cells or support cells in combination with choroid plexus cells. The number of cells or capsules that are implanted into a patient to give a therapeutic effect can vary, for example depending on the interior dimensions of the site of implantation in the body. Typically, if the composition is to be implanted into the cochlea, between 1 and 100 capsules may be implanted. As will be appreciated, this will depend on the dimensions of the capsules, so that for capsules of 700 microns diameter, approximately 50 capsules may be implanted, but for smaller capsules, for example those of approximately 350 micron diameter, up to about 100 capsules may be implanted.

In any event, a physician, or skilled person, will be able to determine the actual number of choroid plexus cells or of capsules containing choroid plexus cells which will be most suitable for an individual patient. This is likely to vary with age, weight, sex and response of the particular patient to be treated. The above mentioned amounts are exemplary of the average case and can, of course, be varied in individual cases.

Implantation of the compositions of the invention requires access to the structures of the middle and inner ear of the recipient. Surgical techniques to gain access to the cochlea or other structures of the middle or inner ear are well known. Techniques for the surgical approach to the human cochlea are described in, e.g., Clark, G M, et al., (1984) and Clark, G M, et al., (1991).

In a further example, a method to allow the placement of a cannula suitable for delivery of the implantable composition of the present invention is described in Gillespie, L N, et al., 2003). Briefly, subjects are anesthetized and SNHL (e.g., ototoxin-induced deafness) is confirmed. Under aseptic conditions, a postauricular incision is made and the left tympanic bulla exposed. The bulla is opened and the basal turn of the cochlea is visualized under a microscope. A fine probe is used to make a pinhole cochleostomy in the scala tympani at the level of the basal turn, and the tip of the infusion cannula is introduced into the hole until the silicone bead rests against the otic capsule, sealing the opening. The cannula is secured in place with Durelon dental cement (ESPE) and two dissolvable sutures. The cannula can then be used to implant the composition of the present invention, or can (as in Gillespie et al., 2003), be connected to a pump, after which the pump may be implanted in a subcutaneous pocket between the scapulae, and the wound is closed with interrupted silk sutures.

An alternative surgical technique suitable for use in the methods of the present invention is described in Lu, W, et al., (2005). Briefly, subjects are anesthetized and a postauricular incision is made following application of local anesthetic. The bony bulla is exposed, and the dorsal region drilled using a high-speed cutting bur. A cochleostomy is performed with a hand drill incorporating an implant quality stainless steel trocar Kirschner Wire (d=0.8 mm) over the round window promontory. Bone chips are removed where possible, and the electrode array is then carefully inserted into the scala tympani. The opening of the cochleostomy is sealed with muscle. For chronic applications, the connector is fixed in the bulla using bone cement (Durelon®, ESPE Dental AG, Germany) and the leadwire assembly fixed to the skull using polyethylene mesh (Lars Mesh, Meadox Medicals, New Jersey, USA).

The placement of a cochlea implant incorporating a drug delivery system is described in Shepherd, R K, et al., (2002). Briefly, prior to injection molding, a length of polyimide tubing (I.D.=0.124 mm; O.D.=0.163 mm; Cole-Parmer Instruments, IL, USA) is placed longitudinally within the central core of the cochlear implant electrode array. After the injected silicone has cured, any protruding polyimide tubing at the apical tip of the array is removed. The opposite end of this polyimide tubing exits the leadwire and is connected to an osmotic pump. The electrode array is connected to a Teflon-insulated multi-stranded stainless steel leadwire connector (seven-stranded, Teflon-coated stainless steel wire; AOM System, WA, USA). The stainless steel leadwire system provides external access to the electrodes for stimulation and impedance measurements (Xu et al., 1997).

Subjects are implanted using sterile surgical techniques. Local anesthetic (e.g., 2% lidocaine) is injected into the wound site. The round window is exposed via a ventral approach, the round window membrane carefully incised with a sterile 25-G needle and the electrode array inserted ˜4.5 mm into the scala tympani. The round window is then sealed with muscle and the leadwire assembly and cannula fixed to the skull using polyurethane mesh and bone cement. The leadwire assembly exits the skin through a small incision placed between the scapulae. Finally, a subcutaneous tissue pocket is created over the left scapula; the end of the PVC cannula is cut and connected to a primed mini-osmotic pump.

This surgical approach is suitable for the implantation of the implantable composition of the present invention. Furthermore, this approach may be used in combination therapies in which the implantable compositions of the present invention are implanted together with a cochlear implant.

Sites in the inner ear other than the scala tympani are suitable for the implantation of the implantable composition of the present invention. For example, the capsules may be placed adjacent to the round window, using a surgical method as described above, or as described in Noushi et al., (2005).

In addition, the “naked” or encapsulated choroid plexus cells, together with any neurotrophin-secreting cells, and optionally support or feeder cells may be introduced into an implantable device before transplantation into a patient. For example, encapsulated choroid plexus cells may be incorporated within or on the surface of a cochlea implant. In one embodiment, the implant device is cell-impermeable but protein or secreted factor-permeable, and may be functionally equivalent to the “TheraCyte™” device (TheraCyte, Inc., Irvine, Calif., USA). As described above, it will be appreciated that the dimensions of the target site must be considered, and accordingly an implantable device must be suitable proportioned for implantation in the middle or inner ear. Alternatively, the choroid plexus cells, and optionally the neurotrophin-secreting cells, the support cells or feeder cells, may be incorporated or embedded in a support matrix which is host recipient compatible and which degrades into products which are not harmful to the host recipient. Natural or synthetic biodegradable matrices are examples of such matrices. Natural biodegradable matrices include collagen matrices. Synthetic biodegradable matrices include synthetic polymers such as polyanhydrides, polyorthoesters, and polylactic acid. These matrices provide support and protection for the cells in vivo. Again, the dimensions of the target site must be considered when constructing the support matrix.

It is envisaged that once implanted, compositions used in the methods of the present invention will be effective for between a few weeks to several months and possibly up to two or more years. The efficacy of the implanted composition can be monitored over time by monitoring one or more factors that are known to be secreted by the choroid plexus cells, or by hearing tests to monitor the function of the auditory nerve or the viability of the SGNs or hair cells, and thus the maintenance of a non-SNHL status in the patient. Should the efficacy of the implantable composition decline, it may be retrieved and replaced by a freshly prepared composition. Such retrieval and replacement of the therapeutic implantable composition may be carried out as often as necessary as part of the treatment regimen to maintain the therapeutic effect.

The main patient group that it is envisaged that will benefit from the present invention are those patients suffering from SNHL. SNHL may be congenital or acquired. Causes of congenital SNHL include a lack of development (aplasia) of the cochlea, certain chromosomal syndromes (rare), congenital cholesteatoma, squamous epithelium hyperplasia, and delayed familial progressive SNHL. Acquired causes of SNHL include inflammatory causes, such as suppurative labyrinthitis, meningitis, mumps, measles, viral agents, and syphilis, exposure to ototoxic drugs, including aminoglycosides (the most common cause; e.g., tobramycin, kanamycin, gentamycin), loop diuretics (e.g., furosemide), antimetabolites (e.g., methotrexate), salicylates (e.g., aspirin), exposure to loud noises (>90 dB), which causes hearing loss beginning at 4000 Hz (high frequency), presbycousis (also referred to as presbycusis or presbyacusis), an age-related hearing loss that occurs in the high frequency range (4000 Hz to 8000 Hz), sudden hearing loss including idiopathic hearing loss, vascular ischemia of the inner ear or cranial nerve 8, perilymph fistula, usually due to a rupture of the round or oval windows and the leakage of perilymph, autoimmune reactions, or Ménière's disease, which is characterized by sudden attacks of vertigo lasting minutes to hours preceded by tinnitus, aural fullness, and fluctuating hearing loss. SNHL is frequently associated with degeneration of hair cells—the ciliated epithelium responsible for transduction of sound in the basilar membrane—and associated degeneration of auditory nerve fibers, called sensorineural hearing loss, and it has been proposed that the decreased stimulation by the functionally diminished hair cells contributes to the degeneration of the SGNs.

Accordingly, the present invention is envisaged to be of benefit to those exposed to ototoxic agents or bacterial and viral agents known to damage hair cells or SGNs, those undergoing Cisplatin treatment, and those acutely or chronically exposed to loud noise.

In addition, patients who are at risk of developing SNHL, for example, children with a family history of SNHL, sufferers of Ménière's disease, or those for whom degeneration of the hair cells or SGNs has been diagnosed may benefit significantly from the present invention.

The present invention is directed to the prevention or treatment of SNHL, via stabilization and preservation of the SGNs, or of the hair cells. In patients, such as those who have already been diagnosed, the present invention aims to deter further SGN or hair cell degeneration.

It is also contemplated that the present invention will be useful in combination with traditional SNHL treatment regimen, such as cochlear implantation. However, it is expected that a significant improvement in SGN or hair cell function would be observed in patients who received the choroid plexus cell containing implantable compositions of the invention.

Accordingly, the invention provides an implantable composition comprising encapsulated isolated choroid plexus cells, preferably porcine choroid plexus cells, which are suitable for administration to a xenogeneic recipient. The implantable composition can be used to treat SNHL, or to delay or prevent the onset of SNHL. The implantable composition used in the present invention may further comprise isolated feeder cells or support cells such as Sertoli cells or fibroblasts.

As used herein, the term “isolated” refers to cells which have been separated from their natural environment. This term includes gross physical separation from the natural environment, e.g., removal from the donor animal, and alteration of the cells' relationship with the neighboring cells with which they are in direct contact by, for example, dissociation.

As used herein, the term “porcine” is used interchangeably with the term “pig” and refers to mammals in the family Suidae. Such mammals include wholly or partially inbred pigs, preferably those members of the Auckland Island pig herd, which are described in more detail in Applicants' co-pending PCT Intl. Pat. Appl. No. PCT/NZ2006/000074 (published as WO2006/110054, and specifically incorporated herein in its entirety by express reference thereto).

The term “treating” as used herein includes reducing or alleviating at least one adverse effect or symptom of SNHL, including impaired hearing or profound hearing loss. The term “treating” as used herein further includes reversing, preventing, or delaying auditory cell degeneration, particularly in patients suffering from or predisposed to SNHL.

As used herein the term “auditory cell” includes cells associated with the generation and transduction of auditory signals, and includes spiral ganglion neurons, the cells comprising the auditory nerve, and hair cells.

Accordingly, the choroid plexus cells, and optionally the neurotrophin-secreting cells, the support cells or feeder cells, are transplanted into a patient suffering from or predisposed to SNHL, in an amount such that there is at least a partial reduction or alleviation of at least one adverse effect or symptom of the disease, disorder or condition, or a reversing, prevention, or delay in auditory cell degeneration.

As used herein the terms “administering,” “introducing,” “implanting,” “transplanting,” and grammatical variants thereof are used interchangeably and refer to the placement of the choroid plexus cells into a subject, e.g., a xenogeneic subject, by a method or route which results in localization of the choroid plexus cells at a desired site. The choroid plexus cells can be administered to a subject by any appropriate route which results in delivery of the cells to a desired location in the subject where at least a portion of the cells remain viable. These administrations will typically be via surgical methods as described herein. It is preferred that at least about 5%, preferably at least about 10%, more preferably at least about 20%, yet more preferably at least about 30%, still more preferably at least about 40%, and most preferably at least about 50% or more of the cells remain viable after administration into a subject. The period of viability of the cells after administration to a subject can be as short as a few days, to as long as a few weeks, to months or years. Methods of administering, introducing and transplanting cells or compositions for use in the invention are well-known in the art. Cells can be administered in a pharmaceutically acceptable carrier or diluent.

The term “host” or “recipient” as used herein refers to mammals, particularly humans, suffering from or predisposed to sensorineural hearing loss into which choroid plexus cells, preferably of another species, are introduced or are to be introduced.

The term “comprising” as used in this specification means “consisting at least in part of.” When interpreting each statement in this specification that includes the term “comprising,” features other than that or those prefaced by the term may also be present. Related terms such as “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### EXAMPLES

The following examples are included to demonstrate illustrative embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation of Encapsulated Choroid Plexus (CP) Cells

This example relates to the preparation of choroid plexus cells suitable for encapsulation and implantation.

### Isolation of CP Cells

Neonatal pigs were anaesthetized with ketamine (500 mg/kg) and xylazine (0.15 mg/kg) and killed by exsanguination. The brain was immediately removed and dissected through the midline to reveal the fork of the choroid vessels. The choroid plexus was extracted and placed in Hanks Balanced Salt Solution (HBSS, 0-4° C.) supplemented with 2% human serum albumin. The tissue was chopped finely with scissors, allowed to settle and the supernatant removed. Collagenase (Liberase®, Roche, 1.5 mg/ml, in 5 ml HBSS at 0-4° C.) was added and the chopped tissues mixed, allowed to sediment at unit gravity (1×g) and the supernatant was again removed. Collagenase (1.5 mg/ml, in 15 ml HBSS at 0-4° C.) was added and the preparation warmed to 37° C. and stirred for 15-20 min. The digested material was triturated gently with a 2-ml plastic Pasteur pipette and passed through a 200-μm stainless steel filter.

The resulting neonatal pig preparations were mixed with an equal volume of RPMI medium supplemented with 2-10% neonatal porcine serum (prepared at Diatranz/LCT). The preparations were centrifuged (500 rpm, 4° C. for 5 min), the supernatant removed and the pellet gently re-suspended in 30 ml RPMI supplemented with serum. This procedure produced a mixture of epithelioid leaflets or clusters of cells, about 50-200 microns in diameter, and blood cells. Blood cells were removed by allowing the mixture to sediment at unit gravity for 35 min at 0-4° C., removing the supernatant and re-suspending. The preparation was adjusted to approximately 3,000 clusters/ml in RPMI with 2-10% serum, and placed in non-adherent Petri dishes. Half of the medium was removed and replaced with fresh medium (5 ml) after 24 hrs and again after 48 hrs. By this time, most clusters assumed a spherical, ovoid or branched appearance.

The cells were then encapsulated in alginate as follows:

### Encapsulation of CP Cells

A counted sample of choroid plexus clusters was washed twice in HBSS supplemented with 2% human serum albumin and once in normal saline. The majority of supernatant was removed from above the sedimented clusters and alginate (1.7%) added in the ratio 1 ml per 40,000 clusters. The clusters were carefully suspended in alginate and pumped through a precise aperture nozzle to produce droplets which were displaced from the nozzle by either controlled air flow (an “air knife”) or by an electrostatic potential generated between the cell suspension exiting the nozzle and the receiving solution.

The stirred receiving solution contains sufficient calcium chloride to cause gelation of the droplets of alginate and cell cluster mixture. After the suspension has passed through the nozzle and the droplets collected in the calcium chloride solution, the gelled droplets were coated sequentially with poly-L-ornithine (0.1% for 10 min), poly-L-ornithine (0.05% for 6 min) and alginate (0.17% for 6 min). The gelled droplets were then treated with sodium citrate (55 mM for 2 min) to remove sufficient calcium from the interior of the gelled capsules to liquidize the contents. The poly-L-ornithine provides sufficient bonding for the capsule wall to remain stable.

The characteristics of the capsules thus produced were reproducibly of 500-700 microns in diameter (98-100%), and were spherical (less than 2% are elliptical or otherwise misshapen). There were few broken capsules (less than 1%). Empty capsules, containing no CP clusters were typically less than 15%. The majority of the cell clusters within the capsules were 100-300 microns along their longest axis. Small clusters (less than 100 microns) were typically 5-13% and large clusters (greater than 300 microns along their longest axis) represented approximately 1-4% of the total.

After encapsulation the cell clusters were more than 90% viable as determined by acridine orange/propidium iodide staining.

### Example 2

### Isolation and Encapsulation of Neurotrophin-Secreting Schwann Cells

This example relates to the preparation of neurotrophin-secretory Schwann cells suitable for encapsulation and implantation.

### Isolation of Schwann Cells

Schwann cells were isolated from the sciatic nerve of postnatal day 2-3 rats. A sub-population of Schwann cells were genetically modified using the lipid-based transfection reagent Lipofectamine 2000 (Invitrogen) to over-express the neurotrophin BDNF. The Schwann cells, both normal and genetically modified, were grown to confluence over 2-5 days on poly-L-lysine-coated cell culture flasks in Dulbecco's modified Eagle's medium (DMEM) containing 2 mM L-glutamine, 50 U/mL penicillin/streptomycin, 10% FCS, 10 ng/ml glial growth factor and 2 μM forskolin, at 37° C., 10% CO₂, and then treated with trypsin and mechanical disruption. The trypsin was inactivated with DMEM containing 2 mM L-glutamine, 50 U/mL penicillin/streptomycin and 10% FCS, and cells were removed from the flask, washed and resuspended at a known concentration prior to encapsulation.

### Encapsulation of Schwann Cells

Encapsulation was carried out using the air knife method essentially as described above. The cells, single or in small clusters (<60 microns), were suspended in alginate (1.7%). The mixture of cells and alginate was pumped vertically downwards through a fine nozzle and the droplets produced were impelled downwards by a concentric air flow. The droplets descended into a solution of calcium chloride (1.2%), became gelled into spheres by the cross-linking action of the calcium ions and settled to the bottom of the solution.

These gelled spheres were washed and serially coated with poly-L-lysine (0.1%, and 0.05%). The poly-L-ornithine provides a polymeric counter-ion to the surface ions of negatively charged carboxyl groups, binding the surface into a tough membrane. The excess charge of the poly-L-ornithine on the outer surface was in turn quenched by a final coat of alginate (0.17%). The formed capsules were then washed in saline and treated with sodium citrate, a mild calcium chelator that liquefied the intracapsular alginate, producing the finished capsule.

Using this method, it is possible to harvest capsules of different size by regulating the speed of the concentric air flow and subsequently by passing the capsules of mixed size through sterile sieves of different mesh size.

### Development and Viability of Encapsulated Schwann Cells

The Schwann cells within the capsules were free to move in the liquefied alginate and form irregular groups that are loosely adherent to each other. Within 24 hr of culture the clusters assumed a spherical appearance. The small clusters often merged with one other, displaying a transiently irregular shape that resolved to a sphere within 24-48 hrs.

Following encapsulation, the cells remained proliferative and viable to 99%, demonstrating an obvious increase in cell number. Viability over 30 days was established to be 98% using the live/dead assay, ethidium homodimer/calcein (available from Molecular Probes, Oregon, USA). FIG. 2 illustrates seven encapsulated Schwann cells maintained in culture for one month post-encapsulation.

### Example 3

### Microencapsulation of Choroid Plexus Cells

This example relates to the preparation of microcapsules containing choroid plexus cells suitable for implantation into the cochlea.

### Isolation of Cells

Choroid plexus cells were isolated as described supra.

### Encapsulation

Microcapsules of 350-400 microns diameter containing choroid plexus cell clusters or Schwann cells were prepared for cochlear implantation using the air knife method as describe above, with the following variations. The concentration of sodium alginate was increased to 1.8%. The cell/alginate suspension was passed through a 23-Ga needle in the air-knife encapsulator at a higher airflow rate of 2.3 L/min.

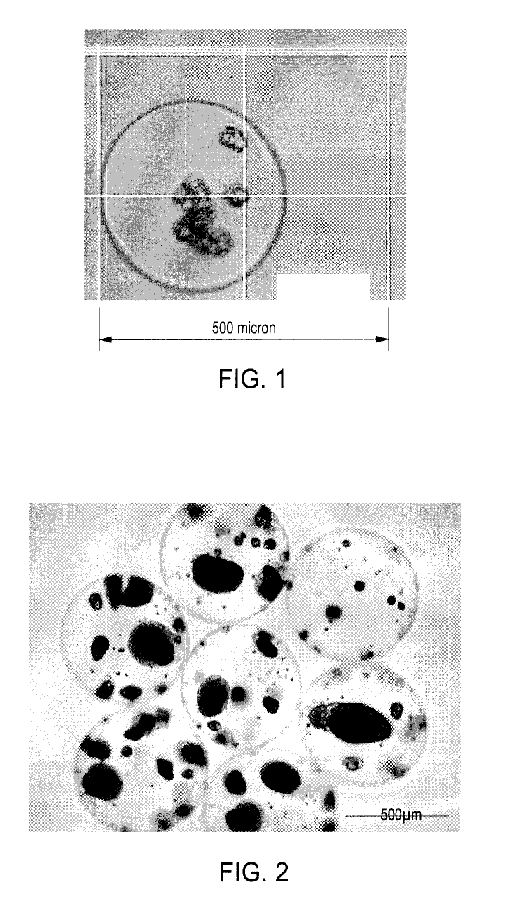

A single microcapsule of approximately 320 microns prepared in accordance with this method and containing choroid plexus cells is shown in FIG. 1.

These studies recognized that there are various potential transplantation sites within the cochlea, all with varying dimensions. For example, the scala tympani, a preferred delivery site within the cochlea for microcapsules of the present invention, diminishes in size as it runs apically from the round window. By controlling the dimensions of the capsules to fit the dimensions of the target site it is possible to deliver capsules of graded size, and therefore to deliver more capsules and more cells. Without wishing to be bound by any theory, this may further extend the benefits of capsule implantation from a local effect to a more generalized effect over the whole cochlea.

### Example 4

### Implantation of Choroid Plexus Cells into the Cochlea

This example relates to the implantation of encapsulated choroid plexus cells into the cochlea of a guinea pig. The results of these studies show that microcapsules prepared as described herein containing choroid plexus cells can be successfully implanted into the cochlea. These data also demonstrated that microcapsules prepared using the methods described herein can remain intact and localized to the implantation site immediately after implantation.

Method of Implantation into the Cochlea

The animal model for implantation used herein is the pigmented guinea pig, a well-characterized and routinely used animal model for SNHL.

### Surgery

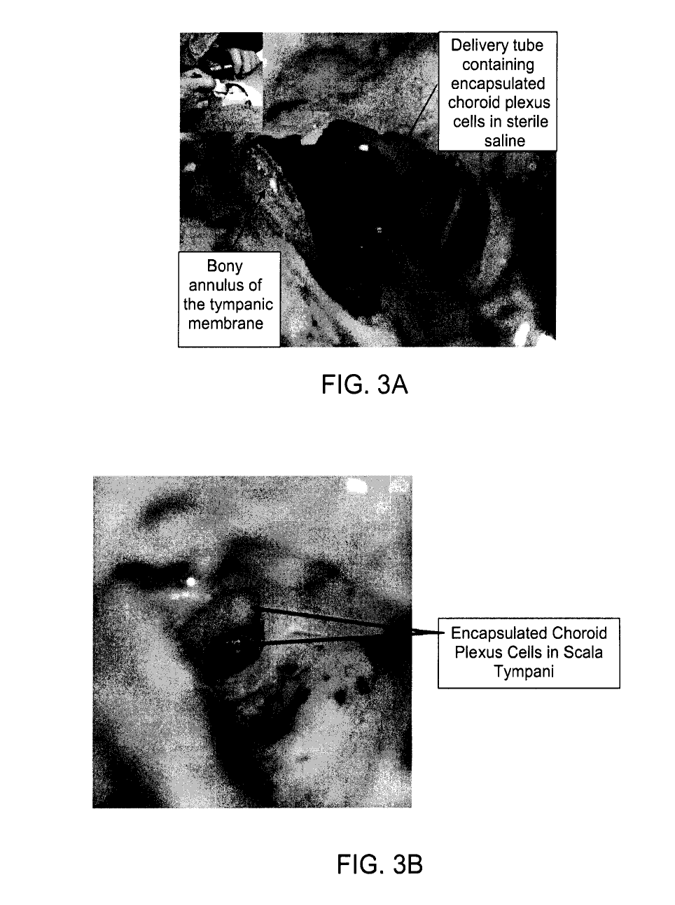

The cochlea of the surgical subject (a 618-gr female guinea pig) was exposed with a postauricular approach via the middle ear to gain access to the basal turn (see FIG. 3A, inset). A delivery tube was inserted into the cochlea and microcapsules containing choroid plexus cells (prepared as described supra and suspended in sterile saline) were infused (FIG. 3A).

FIG. 3B shows the choroid plexus cell microcapsules implanted in the scala tympani of the cochlea.

### Example 5

### Histological Analysis of Implanted Encapsulated Neurotrophin-Secretory Cells

This example demonstrates that encapsulated neurosecretory cells can be implanted atraumatically into the cochlea.

### Methods

The isolation and encapsulation of neurosecretory cells was performed as described herein. Similarly, the implantation of the neurotrophin-secretory cells into the cochlea of a guinea pig was performed as described herein. Cochlea were decalcified and embedded in OCT freezing medium for sectioning. Frozen sections were heated to 37° C. overnight prior to H & E staining.

### Results

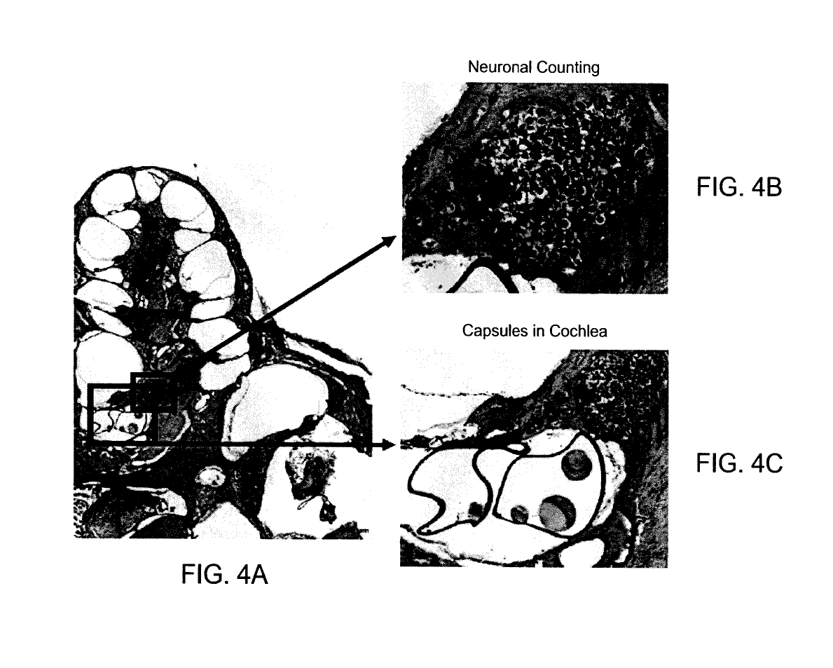

FIG. 4A, FIG. 4B, and FIG. 4C are photomicrographs of a counterstained section showing implanted capsules located in the scala tympani of the guinea pig cochlea. These images confirm that the capsules were atraumatically inserted into the cochlea using the surgical techniques described herein.

As will be appreciated, the histological techniques described above demonstrate that the implantable compositions of the invention can be implanted into a patient in need thereof with minimal deleterious effect. Furthermore, these techniques allow a quantitative assessment of auditory nerve survival, for example by counting the number of surviving auditory neurons. For example, auditory nerve survival can be determined by measuring the density of auditory neuron soma per mm². Neuron density can be measured by a single observer using reported techniques (see, e.g., Coco A, et al., 2006; Shepherd R K, et al., 1983; Shepherd R K, et al., 2005; and Xu J, et al., 1997). Briefly, in each section, the cochlear turns are identified (basal, middle and apical) and the cross-sectional area of Rosenthal's canal within each turn is measured using NIH Image (http://rsb.info.nih.gov/nih-image/). All neurons with a visible nucleus are then counted and neuron density calculated as cells per square millimeter for each turn.

### Example 6

### Expression of Neurotrophic Factors in Encapsulated Choroid Plexus (CP) Cells

This example demonstrates that many of the genes encoding neurotrophic factors are highly expressed in choroid plexus cells suitable for encapsulation and implantation.

### Methods

CP cells were isolated as described supra. mRNA was isolated using the standard methods.

### Results

The expression of the genes identified in Table 1 was determined in CP cells prepared for encapsulation as described herein. Expression levels were calculated as the logₑ of intensity.

TABLE 1
EXPRESSION OF NEUROTROPHIN GENES IN CP CELLS

| | Expression in | | Expression in |
|---|---|---|---|
| | CP Cell RNA | | CP Cell RNA |
| | (Log e | Neuro- | (Log e |
| Neurotrophin | Intensity) | trophin | Intensity) |
| FGF-9 | 4.38 | VEGF | 10.29 |
| FGF-18 | 3.7 | TGF-β2 | 9.3 |
| LIF neural proliferation | 8.58 | TGF-β3 | 6.7 |
| IGF-2 | 11.8 | TGF-β1 | 5.7 |
| IGF-1 | 7.93 | FGF-2 | 6.93 |
| EGF | 9.04 | Acidic FGF | 5.26 |
| EGF | 8.51 | FGF-12 | 5.16 |

These results clearly demonstrate that genes encoding neurotrophins are highly expressed in CP cells prepared in accordance with the methods of the present invention for encapsulation and implantation.

### Example 7

### The In Vivo Effects of Implanted Choroid Plexus Cells on Cochleal Hair Cells

This example relates to the implantation of encapsulated choroid plexus cells into the cochlea of an animal model of SNHL, and the effect of such implantation on the survival and proliferation of hair cells and the inner ear supporting cells (the progenitors of hair cells).

Method of Implantation into the Cochlea

The animal model for implantation is that described herein in Example 4 supra. Delivery of capsules is also as described herein in Example 4 supra. Empty microcapsules are implanted into control groups, while encapsulated cells (CP cells and a combination of CP cells and neurotrophin secretory cells including Schwann cells and Schwann cells genetically-engineered to express BDNF) are administered to test groups.

### Histology

The number and morphology of inner ear supporting cells and of hair cells are compared between treatment groups and control groups using histological methods well known in the art (see, e.g., Andrew [2003]; and Shepherd R K, et al., [2005]) and as described herein (see, e.g., Example 5, supra).

### Results

An increase in the number of inner ear supporting cells or of hair cells, or an improvement in the morphology of inner ear supporting cells or of hair cells, in the treatment group compared to the control group administered empty microcapsules demonstrates a positive effect of CP cell implantation.

### Example 8

### The In Vivo Effects of Implanted Choroid Plexus Cells on Hair Cell and SGN Survival and Function

This example relates to the implantation of encapsulated choroid plexus cells into the cochlea of an animal model of SNHL and the effect of such implantation on the survival, proliferation and function of hair cells and SGNs.

Method of Implantation into the Cochlea

The animal model for implantation and SNHL is a rat model as described herein (see, e.g., Lu W, et al., 2005). Delivery of capsules is as described herein (see, e.g., Example 4, supra). Control groups comprise normal hearing controls, and deafened controls into which empty microcapsules are implanted, while encapsulated cells (CP cells and combinations of CP cells and neurotrophin secretory cells including Schwann cells and Schwann cells genetically-engineered to express BDNF) are administered to test groups.

### Histology

The otoprotective capability of implanted CP cells or combinations of CP cells and neurotrophin secretory cells are assessed by quantifying cell survival and maintenance of neurite innervation with confocal microscopy of fixed tissue. Cochlear slices are taken from treatment and control rats at the onset of hearing at 10 days after birth as described (Jagger D J, et al., 2000), fixed and analyzed using confocal microscopy.

### Function

Assessments of auditory brainstem responses and distortion product otoacoustic emissions are performed on treatment and control groups before and after noise deafening, using techniques well known in the art (see, e.g., Andrew, 2003; Shepherd R K, et al., 2005). These assessments are repeated post implantation, and periodically over the following weeks.

### Results

Hair cell and spiral ganglion neuron counts are performed. Measurements of integrated hearing and hair cell specific indices of temporary and permanent threshold shifts are made, and comparisons between treated groups and control groups (normal hearing, deafened, and ‘empty biocapsule’) are analyzed. An increase in the number of inner ear supporting cells or of hair cells, or an improvement in the morphology of inner ear supporting cells or of hair cells, in the treatment group compared to control groups (normal hearing, deafened+empty microcapsules) demonstrates a positive effect of CP cell implantation on auditory cell survival. An improvement in integrated hearing or in threshold indices in treatment groups compared to control groups demonstrates a positive effect of CP cell implantation on auditory cell function.

### Example 9

### Co-Implantation of Encapsulated Neurotrophin-Secretory Cells and Cochlear Implant Electrode Array

This example demonstrates that encapsulated neurosecretory cells of the invention can be implanted in conjunction with a cochlear implant electrode array device.

### Methods

The isolation and encapsulation of neurosecretory cells was performed as described herein. Similarly, the implantation of the cochlear implant electrode array device was performed as described herein.

### Results

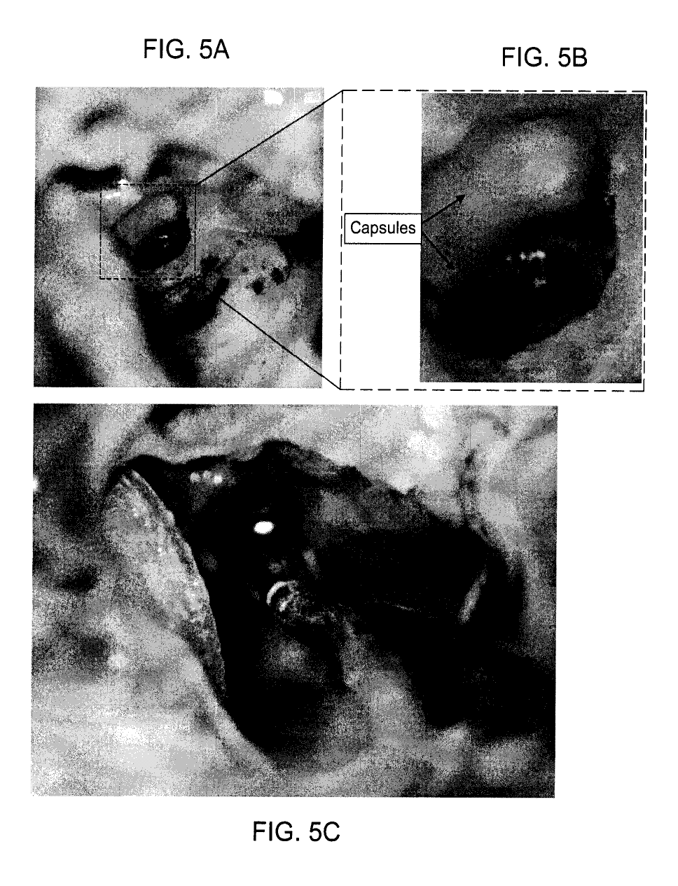

FIG. 5 is a photomicrograph of the surgical delivery of capsules to the cochlea of a guinea pig following the implantation of a cochlear electrode array device. This demonstrates that it is surgically feasible to deliver capsules into a cochlea containing a cochlear implant electrode array. For scale, note that the capsules and the diameter of the electrode array are 0.5 mm.

Given the fact that the human cochlea is significantly larger than the guinea pig, this experiment clearly demonstrates that the delivery of encapsulated cells of the invention together with the implantation of a cochlear electrode array device in the human is feasible.

### Example 10

### The In Vivo Effects of Implanted Choroid Plexus Cells and Cochlear Electrode Array Device Implants on Primary Auditory Neurons

This example relates to the implantation of encapsulated choroid plexus cells into the cochleas of neonatally deafened cats, in the presence or absence of a cochlear electrode array, so as to assess the effect of implanted choroid plexus cells on SGN survival and function. The results of these studies show that microcapsules prepared as described herein containing choroid plexus cells, both alone and in combination with electrical stimulation via the cochlear implant, are able to improve SGN survival and function in this long-term deafened animal model.

### Methods

Cats in the treatment groups (n=18) were neonatally deafened as described in Leake et al., 1991; and Fallon et al., 2009. Neonatally deafened cats were implanted at 8 weeks of age with the capsules (encapsulated choroid plexus cells, n=12; control (empty capsules), n=6) and a multichannel intracochlear electrode array. There were seven normal hearing control animals. Twelve cats received chronic ICES via a clinical cochlear implant and speech processor for a six month period and the remaining six animals did not receive chronic ICES. In order to assess functional changes in electrical thresholds over time as a consequence of treatment, electrically evoked auditory brainstem responses (EABR) were measured every month for each animal as described in Shepherd, R K, et al., 2005.

All cochleae were harvested and prepared for histological examination. Frozen sections of the cat cochlea were cut on the cryostat and produced high quality sections. SGN survival was quantified using blind methods in order to determine the efficacy of treatment by calculating SGN density within the cochlea (n=50).

### Experimental Results

Histological analysis of cochlea taken from the neonatally deafened cat and from control cats was performed as described above. In the normal cochlea, the cell bodies occupy most of the fluid-filled space within Rosenthal's canal. Conversely, there is a substantial decrease in the number of surviving SGNs in the Rosenthal's canal in a deafened cochlea that had received chronic ICES for a period of six months, when compared to the normal cochlea.

### SGN Survival

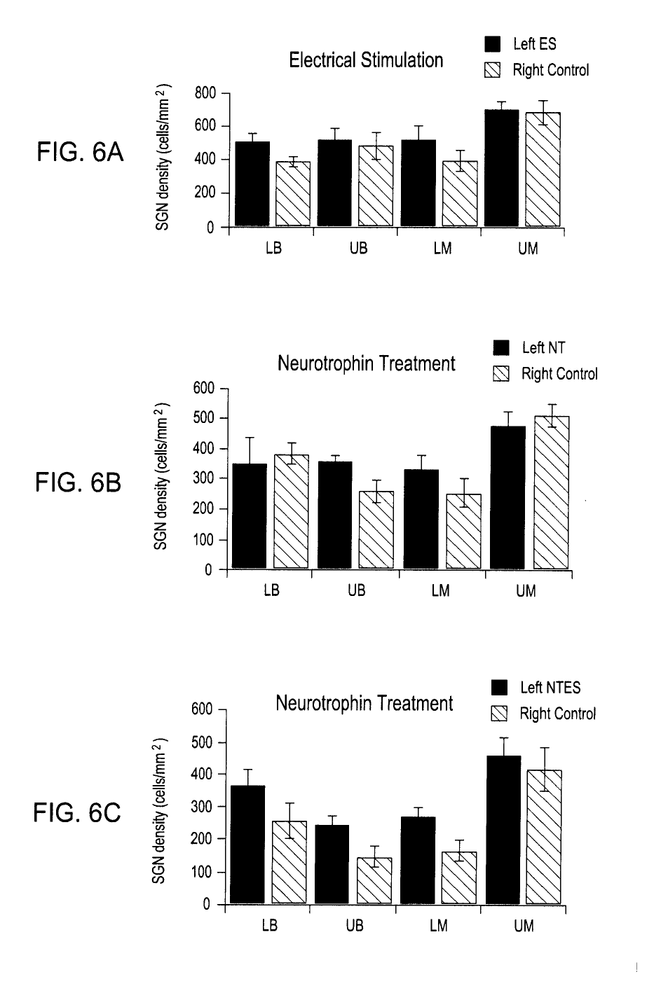

SGN density was assessed as described above and for the three experimental groups data is presented in FIG. 6A, FIG. 6B, and FIG. 6C, as follows: chronic ICES alone (FIG. 6A), encapsulated choroid plexus cells alone (FIG. 6B) and combined chronic ICES and encapsulated choroid plexus cells treatment (FIG. 6C). The results presented in FIG. 6A, FIG. 6B, and FIG. 6C show that chronic ICES alone (with blank capsules) did not significantly improve SGN survival when compared to the contralateral untreated cochlea. Treatment with encapsulated choroid plexus cells capsules alone improved SGN survival in the UB and LM regions of the cochlea, proximal to the site of implantation. Treatment with encapsulated choroid plexus cells capsules alone improved SGN survival in the UB and LM regions of the cochlea, proximal to the site of implantation. Chronic ICES in combination with encapsulated choroid plexus cells was effective in providing a significant increase in SGN survival. There was significantly higher density of SGNs in the lower basal (LB), upper basal (UB) and lower middle (LM) in the cochleae that received the combined chronic ICES and encapsulated choroid plexus cells treatment compared to the untreated contralateral cochlea (RM ANOVA, P<0.003). Approximately 30-40% greater SGN survival was observed in the basal and middle region of the treated cochlea compared to the untreated contralateral cochlea.

### SGN Function

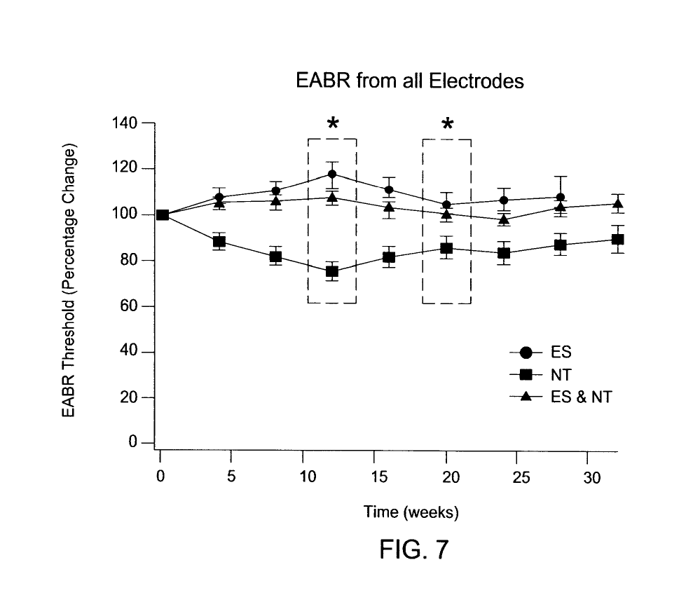

EABR thresholds were assessed as described above for each treatment group. FIG. 7 presents the average of the EABR thresholds represented as a percentage of the value measured in the first recording for each treatment group plotted against treatment period.

Statistical analysis carried out for the thresholds recorded at 12 and 24 weeks indicated that treatment with encapsulated choroid plexus cells only resulted in significantly lower thresholds (ANOVA P<0.003) compared to treatment with chronic ICES.

Accordingly, implantation of choroid plexus cells resulted in a significant reduction in EABR thresholds over time indicative of improved SGN function.

These results indicate that a cell-based therapy can improve SGN function and protect the SGN from death in a clinically viable manner. When used in combination with electrical stimulation from a cochlear implant, a significant and more widespread improvement in SGN survival was observed.

Without wishing to be bound by theory, it is thought that the neurological factors that are secreted by the choroid plexus cells, such as neurotrophin NGF, insulin-like growth factor etc, are involved in maintaining or restoring the viability and function of SGNs and/or hair cells.

It is contemplated that choroid plexus cell implantation will be effective at treating patients who have been diagnosed with SNHL. It is also contemplated that choroid plexus cell implantation will be effective at preventing the degeneration of hair cells or SGNs observed in patients with SNHL.

It is not the intention to limit the scope of the invention to the abovementioned examples only. As would be appreciated by a skilled person in the art, many variations are possible without departing from the scope of the invention as set out in the following indicative claims.

For example, it is contemplated that neurotrophin-secretory cells other than those specifically disclosed herein that have a neurotrophin secretory profile similar to choroid plexus cells will also be useful in the methods of the present invention. For example, cells other than choroid plexus cells that have a neurotrophin factor secretory profile similar to that of choroid plexus cells will also be useful in the methods of the present invention.

### INDUSTRIAL APPLICATION

The present invention is useful in the prevention and treatment of sensorineural hearing loss which will have significant personal, social and economic benefits.

### REFERENCES

The following references, to the extent that they provide exemplary procedural or other details supplementary to those set forth herein, are specifically incorporated herein by reference in their entirety:
Aleshire S L, et al., “Choroid plexus as a barrier to immunoglobulin delivery into cerebrospinal fluid,” J. Neurosurg., 63:593-7, 1985.
Andrew J K, “Rehabilitation of the deafened auditory nerve with Schwann cell transplantation,” BSc Honors Thesis 2003, The University of Melbourne, Melbourne, Australia (as cited in Gillespie and Shepherd, 2005).
Clark G M, et al., “Surgery for an improved multiple-channel cochlear implant,” Ann. Otol. Rhinal Laryngol., 93:204-7, 1984.
Clark G M, et al., “Surgical and safety considerations of multichannel cochlear implants in children”, Ear and Hearing Suppl., 12:15S-24S, 1991.
Coco A, et al., “Does cochlear implantation and electrical stimulation affect residual hair cells and spiral ganglion neurons?” Hear. Res., 225:60-70, 2006.
Coleman, B et al., “Fate of embryonic stem cells transplanted into the deafened mammalian cochlea”, J. Cell Transplant, 15:369-380, 2006.
Fallon J B, Irvine D R, Shepherd R K, “Cochlear implant use following neonatal deafness influences the cochleotopic organization of the primary auditory cortex in cats,” J. Comp. Neurol., 512:101-114, 2009.
Garkavenko O, et al., “Monitoring for potentially xenozoonotic viruses in New Zealand pigs,” J. Med. Virol., 72:338-344, 2004.
Gillespie L K and Shepherd R K, “Clinical application of neurotrophic factors: the potential for primary auditory neuron protection,” Eur. J. Neurosci., 22:2123-2133, 2005.
Gillespie L N, et al., “BDNF-induced survival of auditory neurons in vivo: cessation of treatment leads to accelerated loss of survival effects,” J. Neurosci. Res., 71:785-790, 2003.
Jagger D J, et al., “A technique for slicing the rat cochlea around the onset of hearing,” J. Neurosci. Meth., 104(1):77-86, 2000.
Johanson C E, et al., “Choroid plexus recovery after transient forebrain ischemia: role of growth factors and other repair mechanisms,” Cell Mol. Neurobiol., 20:197-216, 2000.
Leake P A, Hradek G T, Rebscher S J, Snyder R L, “Chronic intracochlear electrical stimulation induces selective survival of spiral ganglion neurons in neonatally deafened cats,” Hearing Res., 54:251-271, 1991.
Lu W, et al., “Cochlear implantation in rats: a new surgical approach,” Hearing Res., 205:115-122, 2005.
Marzella P L and Gillespie L N, “Role of trophic factors in the development, survival and repair of primary auditory neurons,” Clin. Exp. Pharm. Physiol., 29:363-371, 2002.
Noushi F, et al., “Delivery of neurotrophin-3 to the cochlea using alginate beads,” Otol. Neurotol., 26:528-533, 2005.
Sayers S T, et al., “Preparation of brain-derived neurotrophic factor- and neurotrophin-3-secreting Schwann cells by infection with a retroviral vector,” J. Mol. Neurosci., 10(2):143-60, 1998.
Shepherd R K, et al., “A multichannel scala tympani electrode array incorporating a drug delivery system for chronic intracochlear infusion,” Hearing Res., 172:92-98, 2002.
Shepherd R K, et at, “Chronic depolarization enhances the trophic effects of brain-derived neurotrophic factor in rescuing auditory neurons following a sensorineural hearing loss,” J. Comp. Neurol., 486(2):145-158, 2005.
Shepherd R K, et al., “Chronic electrical stimulation of the auditory nerve in cats. Physiological and histopathological results,” Acta Oto-Laryngologica Suppl., 399:19-31, 1983.
Xu J, et al., “Chronic electrical stimulation of the auditory nerve at high stimulus rates: a physiological and histopathological study,” Hear. Res., 105:1-29, 1997.

Although only several exemplary embodiments have been described in detail herein, those skilled in the relevant arts will readily appreciate that many modifications are possible in the exemplary teachings without materially departing from the novel teachings and advantages of this disclosure. Accordingly, all such modifications and alternative are intended to be included within the scope of the invention as defined in the following claims. Those skilled in the art should also realize that such modifications and equivalent compositions, processes, or methods do not depart from the spirit and scope of the present disclosure, and that they may readily make various changes, substitutions, and/or alterations of the compositions herein without deviating from the spirit and scope of the present disclosure.

What is claimed is:

1. A method for stimulating the regeneration of, or delaying the degeneration of, one or more auditory cells in a patient diagnosed with, or at risk for developing, sensorineural hearing loss, the method comprising, implanting into one or both ears of the patient, a cochlear implant device that comprises, distributed over at least a part of an external surface of the device, a population of encapsulated living mammalian choroid plexus cells sufficient to secrete into the one or both ears, an amount of at least one neurotrophin factor and for a time effective to promote the regeneration of, or delay the degeneration of one or more auditory cells in the patient.

2. The method of claim 1, wherein the auditory cells are nerve cells, hair cells, spiral ganglion cells, cochlear epithelial cells, cells of the scala tympani, supporting cells of the organ of Corti, endogenous Schwann cells, or any combination thereof.

3. The method of claim 1, wherein the population of encapsulated living mammalian choroid plexus cells is obtained from a donor porcine or bovine.

4. The method of claim 3, wherein the donor porcine or bovine is a fetal or a neonatal animal.

5. The method of claim 4, wherein the donor porcine is a fetal or neonatal Auckland Island pig.

6. The method of claim 1, wherein the population of encapsulated living mammalian choroid plexus cells is co-cultured with one or more additional mammalian neurotrophin-secretory cells prior to encapsulation.

7. The method of claim 1, wherein the population of living mammalian choroid plexus cells is encapsulated in a biocompatible hydrogel.

8. The method of claim 7, wherein the population of living mammalian choroid plexus cells is support by, or incorporated within, a matrix contained within a biocompatible hydrogel comprising alginate.

9. The method of claim 1, wherein the population of living choroid plexus cells is substantially encapsulated within a population of alginate microcapsules of an average diameter of about 100 to about 700 microns.

10. The method of claim 1, wherein the composition further comprises a population of one or more feeder cells, one or more support cells, one or more neurotrophin-secretory cells, one or more retinal pigmented epithelium cells, one or more dorsal root ganglia cells, one or more Schwann cells, or a combination thereof.

11. The method of claim 10, wherein the population of one or more neurotrophin-secretory cells and the population of living choroid plexus cells are both isolated from the same donor mammal.

12. The method of claim 1, wherein the composition further comprises at least one exogenous neurotrophic factor.

13. A method for treating sensorineural hearing loss in a mammalian patient in need thereof, the method comprising implanting into one or both ears of the mammalian patient, a composition comprising a population of encapsulated living porcine choroid plexus cells sufficient to secrete into the one or both ears an amount of at least one neurotrophin factor and for a time effective to treat sensorineural hearing loss in the mammalian patient.

14. The method of claim 13, wherein the composition is adapted and configured for implantation into a cochlea.

15. The method of claim 13, wherein the composition is adapted and configured for implantation a) at the basal turn of the cochlea; or b) at, or adjacent to, the round window of the patient's inner ear.

16. The method of claim 15, wherein the patient has a cochlear electrode array device or a cochlear implant within the ear.

17. The method of claim 16, wherein the composition is distributed over at least a part of an external surface of the cochlear electrode array device or the cochlear implant.

18. The method of claim 13, wherein the sensorineural hearing loss is congenital, or is caused by age-related hearing loss (presbycusis), Ménière's disease, inflammation, an infection, an autoimmune disorder, trauma, exposure to loud noises, exposure to an ototoxic drug, or any combination thereof.

19. The method of claim 13, wherein the patient is human.

20. The method of claim 19, further comprising fitting the patient with one or more hearing aids.

21. A biocompatible device suitable for implantation into a mammalian ear, the device comprising: a population of encapsulated living mammalian choroid plexus cells capable of secreting effective amounts of one or more neurotrophic factors for a time sufficient to reverse or delay the degeneration of one or more auditory cells, when the device is implanted in an ear of a mammalian patient in need thereof.

22. A cochlear implant device, comprising: a population of encapsulated living mammalian choroid plexus cells distributed over at least a part of an external surface of the device, wherein the population secretes an effective amount of one or more neurotrophic factors when the device is implanted into an ear of a mammalian patient in need thereof, for a time effective to reverse or delay the degeneration of auditory cells in the ear of the patient.
